(19) United States
(12) Patent Application Publication
(10) Pub. No.: US 2005/0240073 A1
(43) Pub. Date: Oct. 27, 2005

(54) Devices and methods to conform and treat body cavities

(76) Inventors: Justus P. Apffelstaedt, Panorama (ZA); Anne DeGheest, Los Altos Hills, CA (US)

Correspondence Address: HELLER EHRMAN LLP, 275 MIDDLEFIELD ROAD, MENLO PARK, CA, 94025-3506, US

(21) Appl. No.: 10/973,808

(22) Filed: Oct. 25, 2004

Related U.S. Application Data
(60) Provisional application No. 60/565,811, filed on Apr. 26, 2004.

(51) Int. Cl. A61N 5/02; A61F 2/00

(52) U.S. Cl. 600/2; 607/100

(57) ABSTRACT

Devices and methods are provided to administer treatment to walls of either naturally occurring cavities or cavities generated by the resection of tissue such as tumors, and to ensure better tissue contact of the device resulting in more effective treatment methods. A device may include a rigid outer surface, that can be brought into firm engagement with the tissue of body cavities by applying suction through channels in the head and stem portion of the device. Methods to treat conformed body cavities tissues may use individual or combination of physical agents including radiation, heat, cold, electrofrequency or chemical agents, such as thrombolytic and cytostatic medications. Systems may include devices described in the invention and sources to provide suction and/or other described means to enhance body tissue conformance to a non-deformable and/or non-distensible body.

### CROSS-REFERENCE TO RELATED APPLICATIONS

This application claims the benefit of U.S. Ser. No. 60/565,811 filed Apr. 26, 2004, which application is fully incorporated herein by reference.

### BACKGROUND

1. Field of Invention

This invention relates generally to methods and devices for applying physical agents such as radiation, heat, cold, electrofrequency or chemical agents, such as thrombolytic and cytostatic medications, to body cavities, including natural body cavities and wound cavities resulting from resection of a tumor, and more particularly to methods and devices to ensure tissue contact and conformance to a non-deformable body introduced to a wound cavity, resulting from resection of a tumor.

2. Description of the Related Art

One method of treating cancer, and breast cancer in particular, is radiotherapy. Conventionally, a “radical” dose of 50 Gy of radiation is delivered to the breast over a period of about one month, and a further five days are required to deliver a so-called booster dose of 10 Gy. In such conventional radiation treatment, the entire volume of the breast is irradiated, and in some cases part of the axilla or parts of the opposite breast are also irradiated. Because of the curvature of the chest wall, it is very difficult to exclude the radio-sensitive tissues of the underlying lung. If one assumes two weeks for surgery and wound healing plus six weeks of radiotherapy, the total treatment time is relatively long at eight weeks.

The reason for irradiating such large volumes of tissue is that it is difficult to locate the tumor bed accurately after closing and healing of the surgical wound and generous margins are necessary to ensure proper coverage of the tumor bed. In addition, it is difficult technically to irradiate a very limited part of the breast due to the constraints of the beam delivery.

An alternative technique which has been used to implant the tumor bed with radioactive iridium wires in theatre, but this has the serious draw-back of increasing theatre time markedly, and exposing the theatre staff to radiation. After placement of the wires, orthogonal x-rays have to be taken in order to determinate the wire positions for dosimetric purposes and the patient subsequently has to remain in a special concrete ward in isolation for several days.

Conventional radiotherapy treatment of the breast may lead to undesirable damage to large areas of tissue, and therefore the total dosage of radiation that can be delivered to the malignant tissue, is limited. A number of radiation techniques are used, including conventional radiotherapy and intraoperative electron therapy. In the latter case, the tumor bed to be irradiated must be exposed and manipulated to accommodate the electron applicator, which is tedious or imprecise. Brachytherapy is a form of intraoperative radiation therapy in which a radiation source is placed in or near the malignant tissue, typically utilizing catheters into which are inserted radioactive wires near to the tumor to be irradiated. For example, U.S. Pat. No. 6,179,766 describes a brachytherapy method for treating breast cancer.

It remains an ongoing problem to be able to control the intensity, distribution and depth of radiation applied to the tissue to be irradiated, while protecting healthy tissue and minimizing the dose of radiation applied to it. It is also desirable to minimize the time required for a course of radiotherapy, and the extent of sedation or anesthesia required to administer the treatment.

A number of inventions have been described, where an expandable or deformable outer surface is used to control the delivery of radiation to a cavity, for example, U.S. Pat. No. 5,429,582 to Williams, U.S. Pat. No. 5,913,813 to Williams et al., U.S. Pat. No. 5,931,774 to Williams et al., U.S. Pat. No. 6,022,308 to Williams, U.S. Pat. No. 6,083,148 to Williams, and U.S. Pat. No. 6,413,204 to Winkler et al., the disclosures of which are all hereby incorporated by reference in their entireties. These devices present major problems in ensuring, that the inflation of the device is uniform with no inclusions e.g. air bubbles and that the device does not undergo conformal changes between implantation and treatment administration, such as for example by perforation and deflation during surgical manipulation. The importance of reliable concentric outer configuration can be illustrated by the fact that during radiation therapy with a point source at the center of the device 2 mm difference in distance from the source may represent the difference between under- and over treatment.

The main intention of applying radiation after surgical removal of a tumor is to render microscopic residual tumor, which is accepted to be the basis of tumor recurrence in the long run, unable to proliferate. As such, a margin of 3 to 10 millimeters is treated with a tumoricidal dosage of radiation to sterilize the tumor bed. While the same reduction in recurrence can be achieved by a wider surgical resection, this is undesirable as it leaves, notably in the case of breast cancer, a cosmetically unacceptable deformity. Other methods of tissue sterilization, which leave volume intact, include the application of physical agents such as heat, cold or radiofrequency or chemical agents such as cytostatic medications.

In methods and devices, where a physical agent is applied during or after surgery in a wound cavity, there is a problem to ensure, that the device is in contact with the wound surface at all times during radiation and not displaced by wound fluids such as hematoma or seroma or insertional artifacts such as air bubbles which would lead to undesirable low dosages delivered to the tissues displaced.

Yet another problem with postoperatively placed devices is placement into the correct position, which is currently estimated by imaging and aiming the device at the putative resection cavity.

There is a need for methods and systems that overcome these deficiencies by presenting a reliable, non-deformable shape with defined, unchanging characteristics for conduct of physical agents and conduct of biologically active materials.

### SUMMARY OF THE INVENTION

Accordingly, an object of the present invention is to provide methods and systems to administer treatment to walls of either naturally occurring cavities or cavities produced by the resection of tissue such as a tumor.

Another object of the present invention is to provide improved methods and systems for conformance of walls of a body cavity to a treatment device at least partially positioned in the body cavity.

A further object of the present invention is to provide methods and systems for conformance of walls of a body cavity to a treatment device at least partially positioned in the body cavity that apply a vacuum to improve conformance.

In one embodiment of the present invention, an application apparatus has a non-deformable body with a head portion shaped configured to be positioned within a body cavities resulting from removal of a tumor. The non-deformable body is sized to receive a treatment agent. A stem portion is coupled to the body and is sized to protrude out of the wound cavity and receive the treatment agent. A conformance member is included that causes tissue in the vicinity of the head portion to become engaged with the head portion.

In another embodiment of the present invention, a method is provided for administering a physical agent to human tissue. A source is located in an applicator body that defines a rigid wound-engaging surface. The applicator body is inserted into a wound cavity to provide that a wound-engaging surface is in contact with tissue defining the wound cavity. A conformance member is utilized to cause tissue in the vicinity of the head portion to become engaged with the head portion. The applicator body is left in position in the wound cavity for a sufficient time to deliver a treatment dose of a physical agent to tissue adjacent the wound cavity. The applicator body is then removed from the wound cavity.

In another embodiment of the present invention, a system is provided for treating walls of either naturally occurring cavities or cavities that are produced by the resection of tissue. A body is included with a head portion that is shaped to be positioned within a body cavity resulting from removal of a tumor. The body is sized to receive a treatment agent. The body has a first channel to receive a physical or chemical agent, and a second channel that extends away from a surface of the head portion. A stem portion is coupled to the body and is sized to protrude out of the body cavity and receive the treatment agent. The first and second channels extend through the stem portion to a coupling on the stem portion remote from the head portion. A vacuum source is configured to operate and connect with at least one of the first and second channels in the stem portion.

### BRIEF DESCRIPTION OF THE DRAWINGS

FIG. 1 is a schematic side view of a human breast showing a carcinoma therein. FIG. 2 is a schematic side view corresponding to FIG. 1, showing a wound cavity remaining in the breast after surgical removal of the carcinoma.

FIG. 3 is a similar view to that of FIGS. 1 and 2, showing the application device of the present invention inserted into the wound cavity.

FIG. 4 is a front view corresponding to FIG. 3, showing the application device in position.

FIG. 5 is a side view of the application device for the present invention.

FIG. 6 is a diagrammatic illustration of the use of the application device with a High Dose Rate, Remotely controlled After-loading Brachytherapy Unit (HDRRCABU) as source of the physical agent.

FIG. 7 is a schematic side view corresponding to FIGS. 1 to 3, illustrating the radiation dosage distribution of the device in use for a single, central main channel.

FIG. 8 is a side view showing the openings of the further channels on the surface of the head portion.

FIG. 9 is a schematic trans-sectional view of the application device with the main channel and the further channels, which extend from the surface of the head portion through the head portion and the stem portion.

FIG. 10 is a schematic drawing illustrating the poor contact between application device and tumor bed.

FIG. 11 is a schematic drawing showing the container in place and an external force, e.g. gas pressure, molding the tumor bed around the application device.

### DESCRIPTION OF PREFERRED EMBODIMENTS

The present invention provides devices and methods to administer treatment to walls of either naturally occurring cavities or cavities generated by the resection of tissue such as tumors. Preferred embodiments include features and methods to improve tissue conformance to the treatment device and ensure better tissue contact of the device resulting in more effective treatment methods.

In one embodiment, methods and systems are provided to improve tissue conformance to the treatment device utilizing a variety of different devices and methods, including but not limited to, vacuum sources devices to ensure better tissue contact of the device resulting in more effective treatment methods. In one embodiment, the methods and systems of the present invention utilize a non-deformable body with a head and a stem portion. The head portion defines a rigid outer surface that can be brought into firm engagement with the tissue of body cavities, including natural body cavities and wound cavities. This locates the source of a physical or chemical agent accurately relative to the tissue. An application apparatus may include a rigid outer surface, that can be brought into firm engagement with the tissue of body cavities by applying suction through channels in the head and stem portion of the device. Methods to treat conformed body cavities tissues can use individual or combination of physical agents including but not limited to, radiation, heat, cold, electrofrequency or chemical agents, such as thrombolytic and cytostatic medications. In one embodiment, a systems of the present invention provides suction and other means to enhance body tissue conformance to a non-deformable and/or non-distensible body.

Engagement with a body cavity surface can be achieved in a variety of ways, including but not limited to, physical devices attached to the surface of the head portion, such as miniature tissue clamps or hooks or vacuum administered through different means such as channels in the head portion, chemical means, such as coating of the head portion with tissue adhesives (Tissue colle®, fibrin glue), enclosing the organ out of which the tumor was resected in a container and exerting a force by e.g. mechanical or magnetic means, to conform the tumor cavity round the head portion, and the like.

In one embodiment, the body is solid and is made of a material that is substantially transparent to ionizing radiation, or conduct other physical agents such as heat, cold, light or electricity or are enhancing the action of other physical agents such as magnetism. The body can be made of other materials, including but not limited to, PTFE or another suitable inert biocompatible materials such as metals or alloys or rapidly bio-absorbable materials such as polygalactin, a fluid permeable mesh such as braid or woven structure of polymeric materials that can provide a non-distensible surface with small openings to allow suctioning and or fluid exchange between the head and the wound surface. Suitable braided materials include nylon and other generally non-distensible polymers, and the like.

In one embodiment, the head portion is spherical or spheroidal with a diameter in the range of 1 to 150 millimeters. The main channels in the body can be tubular and sized to receive guide tubes for the delivery of physical agents. In one embodiment, the main channels extend through and terminate a distance beyond the center of the head portion calculated to position the source of the physical agent at the center of the head portion in use or at other defined points in the head portion. The stem portion can have a coupling provided at an end thereof remote from the head portion that is adapted to receive the guide tube and to clamp it in position relative to the device. The coupling can be compatible with a guide for delivery of a physical agent, including but not limited to a radiation delivered by a conventional High Dose Rate, Remotely Controlled After-loading Brachytherapy unit (HDRRCABU), other radiation sources such as a miniaturized x-ray tube, or other sources of treatment agents such as heat as generated by radiofrequency and cold a generated by e.g. liquid nitrogen.

Further channels in the head portion can be provided, such as tubular geometries, that continue through the stem portion to serve as conduction channels for either vacuum or substance administration to the surface of head portion. The stem portion can have a coupling provided at an end thereof remote from the head portion that is adapted to be connected to a device generating a vacuum or reservoirs for medications such as anti-coagulants. Suitable medications include but are not limited to, heparin or thrombolytic agents, such as tissue plasminogen activator, urokinase or other biologically active substances, e.g., cytostatic or cytotoxic medications to be delivered via the stem portion to the channels in the body and ultimately the surface of the body portion. Flushing can occur concurrently with the radiation, during brief periods before radiation has started, and/or after all radiation has been completed.

The head portion and/or the stem portion may be coated with substances that enhance engagement of the head portion with the surrounding tissue including but not limited to, anti-coagulants, for example heparin, thrombolytic agents, for example tissue plasminogen activator, biocompatible adhesives, for example fibrin glue, and the like. The head portion and/or the stem portion may be wholly or partially coated with or made of substances that enable or enhance the delivery of physical agents such as metals for the delivery of an electrical current. The head portion and/or the stem portion can carry markers on the surface of embedded in the substance that facilitate detection and positioning in the wound cavity by providing optical guides or contrast to imaging modalities such as contrast to x-rays or magnetic resonance investigations.

A container can be used to enclose the organ out of which the tumor has been resected to conform the resection cavity around the head portion of the device by exerting a force from the outside of the organ for the tumor cavity to conform around the head portion of the device. Such force may be generated by several means such as mechanical, liquid, gas pressure or magnetism forces.

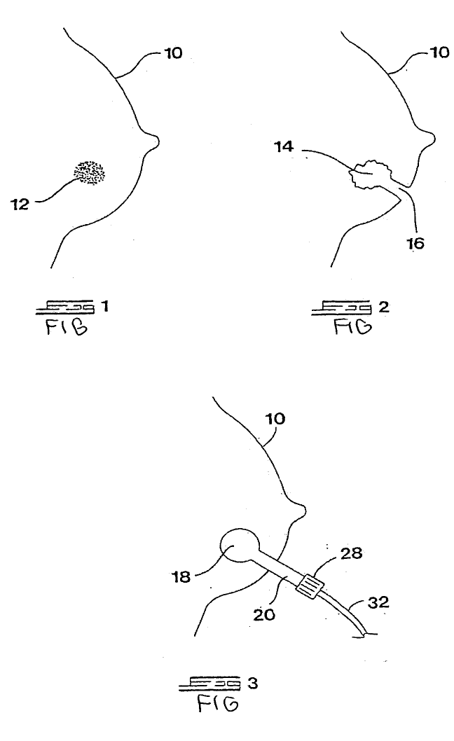

FIG. 1 is a schematic side view of a human female breast 10 in which breast cancer has manifested in the form of a carcinoma 12. FIG. 2 is a similar view to that of FIG. 1, showing a wound cavity 14 that remains in the breast after removal of the carcinoma by way of a so-called “lumpectomy”. Conventionally, the wound opening 16 would be closed by stitching and a course of radiotherapy would then be administered to the general area of the wound or tumor bed.

In example 1 below, an alternative form of postoperative radiation treatment fro breast cancer is provided.

An embodiment of the present invention is shown in FIG. 5, and comprises a bulbous head portion 18 that is preferably spherical or spheroidal, and an elongate stem portion 20, that serves both as a handle to manipulate the device and as a connector for a guide tube that guides a radioactive source into the head of the device. The stem 20 extends radially away form the spherical head 18 and defines a tubular bore of channel 22 that extends into the head 18. the end 24 of the channel is located just beyond the center of the head 18, so that a radioactive point source introduced into the device via the channel 22 will be located as closely as possible to the center of the spherical head 18.

At the end of the stem remote from the head 18 a tapered screw thread is formed, onto which a complementally threaded knurled locking collar or knob 28 can be screwed. The knob has a central aperture 32 that is sized to receive a guide tube 32 of source of a physical agent, such as in example 1 a high Dose Rate, Remotely Controlled After-loading Brachytherapy Unit (HDRRCABU) and to clamp the end of the tube in position once it is correctly attached to the an embodiment of the present invention, by hand tightening the knob onto the thread 26.

The body of the an embodiment of the present invention is preferably manufactured from a single piece of PTFE or “Teflon” (trade mark) or another suitable tissue compatible material that has the required properties, for example of being substantially transparent to ionizing radiation in the case of radiation being used as the physical agent, or being of a conductor for other suitable physical agents, such as heat or laser light. Obviously, the material of the embodiment should be non-reactive and not be toxic or irritating to human tissue. Apart from PTFE, other medical grade plastics materials or metals and alloys should be suitable for the manufacture of the device.

The prototype embodiment had a head 18 that was 50 millimeters in diameter, with a stem or handle 20 that was 100 millimeters long and 15 millimeters in diameter. The channel 22 had a diameter of 3 millimeters and the end 24 thereof extended past the geometric center of the spherical head by approximately 2.5 millimeters.

The head 18 of the device can have a diameter from approximately 1 mm to 150 mm, to cater for different sized wound cavities. It will be appreciated, that this range of sizes is purely exemplary, and the size and also the shape of the head can be adjusted according to the size and nature of the wound cavity and the tumor bed to be treated. For example, the head of the embodiment could be ellipsoid or banana shaped instead of being spherical or spheroidal.

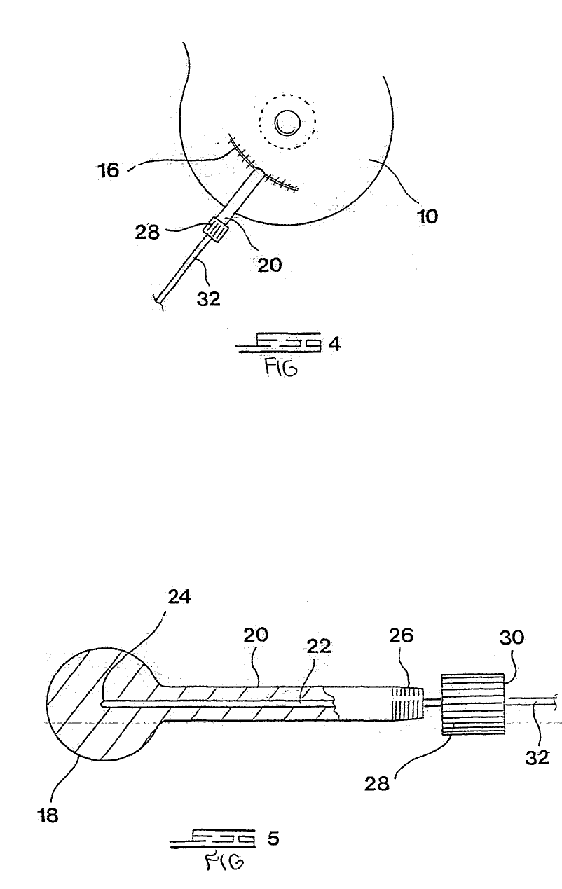

Referring to FIGS. 3, 4 and 6, the radiation application device is shown in use. FIG. 3 shows schematically the device of the present invention being applied to the wound cavity 14. The stem 20 of the device extends from the wound edges 16, that are preferentially stitched closed around the stem so that the head of the device is effectively buried within the wound cavity and an obturator or plug. An embodiment having a suitable head diameter is chosen so that the tissue must be stretched somewhat to ensure firm contact of the wound cavity walls with the rigid outer surface of the head. This has the important result that the position, depth and size of the wound cavity and the tissue to be treated are known with certainty.

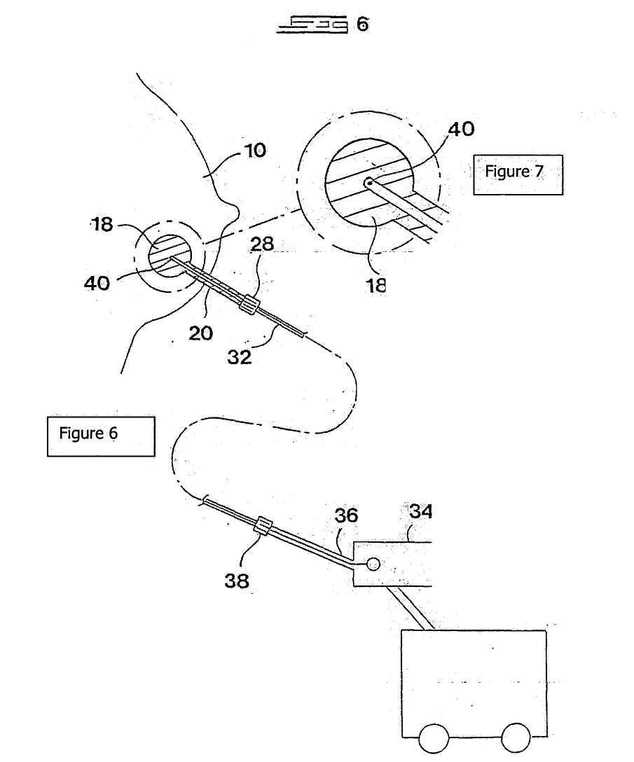

As seen in FIG. 6, in example 1 of the present invention, radiation is chosen as the physical agent and a conventional HDRRCABU 34 is used in conjunction with an embodiment of the present invention. A main guide tube 36 extends from the head of the HDRRCABU 34 and is provided with a coupling 38 that permits the guide tube 32 to be attached thereto. The guide tube 32 will typically a 200 millimeter non-flexible stainless steel tube of approximately 2 millimeter diameter. Instead of a rigid tube, a flexible tube might be preferred in certain cases.

Within the head of the HDRRCABU 34 is a drum on which is wound a length of piano wire or similar stiff wire, with a small radioactive source 40 fixed to the end thereof. The source is preferably cylindrical or spherical and comprises Iridium 192 or another source suitable for irradiation of human tissue, such a miniaturized x-rays tube or linear accelerator. The source is sufficiently small to act as an isotropic point source. The source dimensions of a typical commercially available HDRRCABU are about 0.5 mm in diameter and about 5 millimeters in length. The location of the end 24 of the channel 22 in the head of the prototype device was determined by these dimensions. This would not preclude the use of a spherical isotope source other than 192Ir, for example 37Cs or miniaturized x-rays tubes or linear accelerators.

Returning to example 1 of the present invention, with the guide tube 32 clamped firmly in place between the stem 20 of the embodiment of the present invention and the main guide tube 36 of the HDRRCABU 34, the machine can be operated to drive the wire and thus the radioactive source 40 along the tubes, into the center of the head 18 of the embodiment of the present invention.

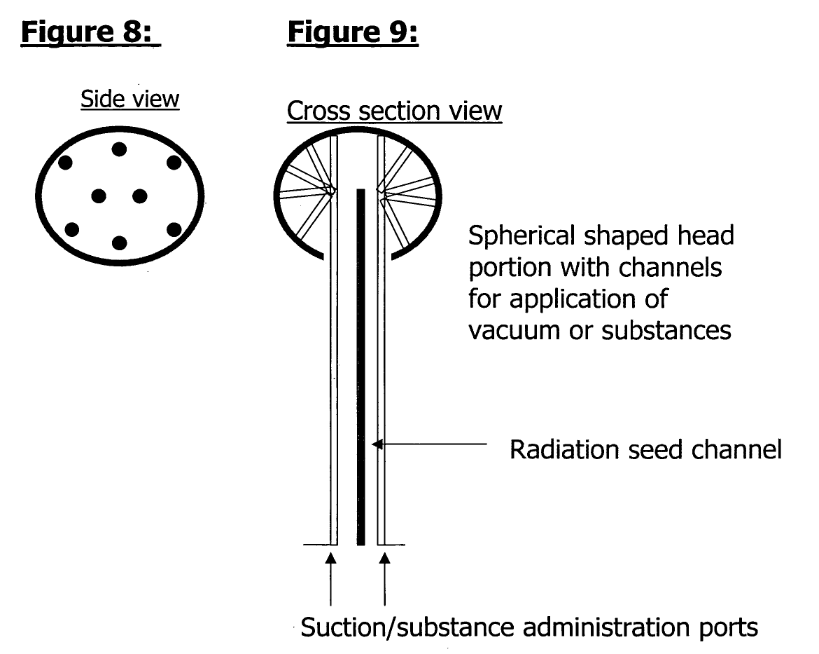

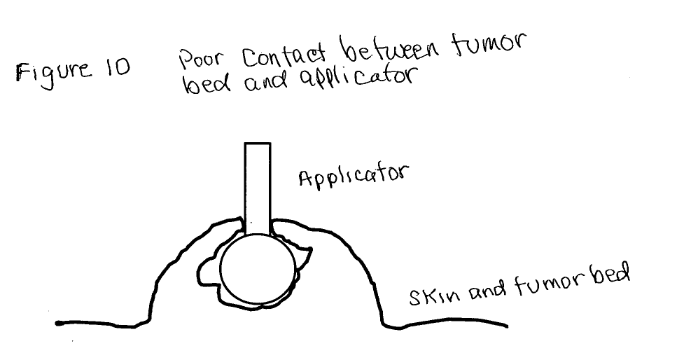

FIG. 7 shows an example of the isotropic radiation intensity/dose distribution due to the radioactive source 40 at the center of the head 18 of the embodiment of the present invention. Assuming that the diameter of the head 18 is 50 millimeters, that is, the radius is 2.5 centimeters, and assuming a dose at the surface of the embodiment of the present invention of 10 gy, the dose 1 centimeter away from the embodiment will be 4.8 gy, and the dose 2 centimeters away will be 2.8 Gy. In a practical application, the radiobiological considerations will be taken into account.

The alpha/beta ratio is a very well tested quantity in the linear quadratic model of radiation damage. This model was first introduced by Lea and Catcheside already in 1942, and shown to be applicable to clinically relevant radiation damage by Dale and co-workers in the United Kingdom and by Orton and co-workers in the United States of America. The damage caused by a particular schedule of irradiation to the cancerous tissue as well as to the normal tissues can be predicted with a very fair degree of confidence by aid of the linear quadratic model. The alpha/beta ratio for cancerous tissue is usually about 7 Gy and for the relevant normal tissues of the breast (excluding the skin) is 2 Gy.

For a dose of 50 Gy delivered in 5 weeks in 2 Gy increments, the Biologically Effective Damage to cancerous tissue and skin is given by

$$\begin{matrix}BED ( 7 ) = nD ( 1 + d / a / b ) Gy \\ = 25 \times 2 ( 1 + 2 / 7 ) Gy \\ = 64.28 Gy .\end{matrix}$$

For the relatively large volume encompassed by the additional “booster” dose, the value for the BED (7) will be 77.14 Gy. It is therefore necessary to calculate equivalent values for the radiation application device of the present invention.

If a single dose of 21 Gy delivered to the surface of the applicator is chosen, then a “shell” of tissue 1 cm thick will receive the following doses:
A: Tissue in contact with the applicator:
   BED(7)=21(1+21/7)Gy=84 Gy

So it can be shown, that at 1 mm from the surface, the BED(7) will be 73 Gy, about the same as a radical dose plus booster dose, and at 2 mm from the surface, the BED(7) will be 64 Gy, or equivalent to the BED(7) of the dose due to full breast irradiation.

At 3 mm from the surface, the BED(7) will be 57.12 Gy and at 5 mm from the surface, 44.89 Gy and at 10 mm from the surface, the BED(7) will be 27.4 Gy (see table below).

### EXAMPLE 1

TABLE 1
Calculation of the volumes treated in example 1 of the present
invention and comparison to traditional treatment:

| | | Volume |
|---|---|---|
| Biologically effective dose to: | | irradiated: |
| 1. | The whole breast = 64.4 Gy | Approximately |
| | | 850 cc |
| 2. | Whole breast plus tumor bed: 77.16 Gy | 600 cc |
| 3. | Shell of breast tissue from the surface of the | 114 cc |
| | applicator of diameter 5 cm: the dose drops from | |
| | 21 Gy surface dose to 26.9 Gy at a radius of 3.5 cm | |
| | and the volume of this irradiated shell of tissue is | |

Therefore volume of breast tissue spared from irradiation: (850−114)cc=736 cc.

The volumes are exemplary and relate to an “average” sized breast. The volume in each case may vary, of course, according to the size of the breast.

### EXAMPLE 2

TABLE 2
Variation of the radiation dose from the surface of a 5 cm diameter
spherical applicator:
Table 2 shows how the dose drops from 21 Gy at the surface of the
embodiment of the present invention to the radii indicated, as well
as the volumes of tissue irradiated to the corresponding dose levels:

| Radius (cm) | Dose (Gy) | BED(7) (Gy) | Volume irradiated (cc) |
|---|---|---|---|
| 2.5 | 21 | 84 | 0 |
| 2.7 | 18 | 64 | 82.5 − 65.5 = 17 |
| 2.8 | 16.8 | 57 | 92.0 − 65.5 = 26.5 |
| 3.0 | 14.5 | 47.5 | 113 − 65.5 = 47.5 |
| 3.5 | 10.8 | 27.4 | 179.7 − 65.5 = 114.2 |

Generally, the method of invention can be used to deliver a radiation dose at the surface of the applicator in the range 5-30 Gy.

Similar considerations are valid for the delivery of other physical agents: In the case of heat generated by radiofrequency, the change in tissue density around an applicator can be observed and measured under real-time ultrasound to indicate the thickness of the shell treated around the applicator of the present invention. The same holds true for cold administered for example by liquid nitrogen probe to an embodiment of the present invention.

The excision of tumors out of any organ in the human body is designed to remove a margin of macroscopically normal (non infiltrated) tissue around the tumor of about 2 cm in thickness. Treating an additional volume of about 50 cc that will still have a significant tumoricidal effect, will add significantly to lowering the chance of local recurrence. Since the treatment effect of physical agents is reduced very fast with distance due to the isotropic nature of the source, the risk of damage to adjacent organs such as lungs, ribs, skin and heart (such a in applications to the left breast) which is problematic in the case of externally applied radiotherapy, is virtually eliminated.

### EXAMPLE 3

TABLE 3
The volumes of surgically removed breast tissue plus the value
sterilized in principle by the local administration of a physical agent,
in this example radiation:

| Tumor | | | Volume surgically |
|---|---|---|---|
| diameter | Volume of | Volume of surgically | removed plus |
| (cm) | tumor (cc) | removed shell | irradiated shell |
| 1 | 0.5 | 65.5 | 182.5 |
| 1.5 | 1.8 | 87.2 | 201.2 |
| 2.0 | 4.2 | 113.1 | 227.1 |
| 2.5 | 8.2 | 143.8 | 257.8 |

Generally, the method of the invention would be suitable to treat a layer of tissue surrounding an embodiment of the present invention of between 0 and 20 millimeters thickness.

Treatment using the method and an embodiment of the present invention can as much as treble the volume of tissue rendered “safe” surgically, yet the irradiated volume is only about 25% of the volume of breast tissue irradiated by the standard current method.

In one embodiment of the present invention, the treatment time comprises the time required to insert the applicator and deliver, for example, a radiation dose of 21 Gy. This should take approximately half an hour. As soon as the radiation dose has been administered, an embodiment of the present invention can be removed and the wound closed. The patient can then be discharged. Thus, a great deal of time and expense can be saved. If the tumor should recur, a re-excision of the lesion as well as irradiation by conventional means is possible, which is of further benefit to the patient.

A number of embodiments of the present invention are possible. For example, instead of being designed for use with a HDRRCABU, one embodiment of the invention can be designed for use with a separate radiation source, which is inserted into the channel 22 and held in position with a suitable plug extending into the channel. The embodiment can then be inserted into a wound cavity as described above, and left in position for a prescribed period. In such a case, due to the handling of the embodiment that would be required with the radiation source in place, lower dose rate therapy would probably be applied in this way, requiring hospitalization of the patient for a few days.

Although the delivery of the required treatment dose as a single dose has been described and is generally preferable, the dose could be delivered in several fractions or dose increments if desired. Since the embodiments of the present invention are rigid, regular in shape and non-deformable, the basic dosimetry is much easier and more reliable than it would be with a deformable applicator. This makes a rigid (solid) embodiment inherently more predictable and safe as compared to a non-rigid one.

An inherent problem of the placement of devices the working of which is relies on good surface contact with the surrounding tissue in wound cavities is the naturally occurring presence and continued accumulation of wound fluids such a blood (which may clot) and serous fluid. Furthermore, air introduced with the device may break the surface contact with the device. The different embodiments of the present invention address this problem by the presence of further channels in the head and stem portion (FIG. 8). Through these channels, a variety of physical and chemical agents can be administered to the surface of the head portion, including but not limited to the following:
(i) Vacuum may be administered through these channels that sucks the surrounding tissue to the surface of the head portion;
(ii) Anti-thrombotic agents such as heparin, may be administered through the channel to prevent blood clot formation around the head portion; and
(III0 Thrombolytic agents such as tissue plasminogen activator may be administered to liquefy existing clots.

Any combination of the above methods may be administered, such as upon detection of a clot first a lytic agent to lyse the clot, then suction to evacuate the lysed clot and then an anti-coagulant to prevent formation of further clots. These approaches combined or separate improves the direct contact between the wound tissues and the surface of the body to ensure radiation is applied to the entire wound surface and to provide better control of the radiation depth. Furthermore, therapeutic agents, such as cytostatic or cytotoxic agents could be administered through these further channels. An example of such an agent is bleomycin.

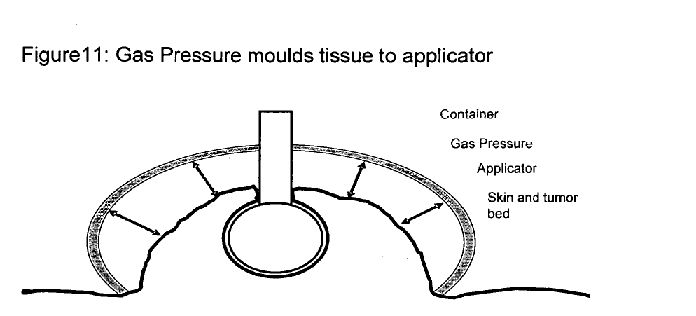

In other embodiments of the present invention, external forces can be used to ensure good contact between the surrounding tissue and the surface of the head portion. One example of an embodiment of the present invention is to use electrical forces by coating the head portion with magnetic material in such a way that a magnetic field can be generated to compress the tissue to conform onto the head body. Another example of an embodiment of the present invention would be to use mechanical forces such as clamps on the surface of the head portion to clamp the tissue to be treated or an outside device compressing the breast externally. Examples of external embodiments could include a cup with an expandable balloon, with air or liquid, on the side of the breast tissue or heavily weighted materials in the shape of the breast to put equal pressure externally on breast tissue (FIG. 11). Another example of an embodiment of the present invention is a sealing enclosure of the organ to be treated and gasses or liquids entered in between the sealing enclosure of the organ to be treated and the surface of the organ to exert a uniform pressure that conforms the tissue to the head portion.

The foregoing description of preferred embodiments of the invention have been presented for purposes of illustration and description. It is not intended to be exhaustive or to limit the invention to the precise forms disclosed. Obviously, many modifications and variations will be apparent to practitioners skilled in this art. It is intended that the scope of the invention be defined by the claims and their equivalents.

1. An application apparatus comprising:
a non-deformable body having a head portion shaped configured to be positioned within a body cavities resulting from removal of a tumor, the non-deformable body sized to receive a treatment agent;
a stem portion coupled to the body and sized to protrude out of the wound cavity and receive the treatment agent, and
a conformance member that causes tissue in the vicinity of the head portion to become engaged with the head portion.

2. The apparatus of claim 1, wherein the body cavity is a natural body cavity or a wound cavity.

3. An application apparatus comprising:
a non-deformable body having a head portion shaped to be positioned within a body cavity resulting from removal of a tumor, the body sized to receive a treatment agent;
a stem portion coupled to the body and sized to protrude out of the wound cavity and receive the treatment agent, and
a conformance member that causes tissue in the vicinity of the head portion to become engaged with the head portion and optimize an amount of tissue treated by the treatment agent.

4. An application apparatus comprising:
a non-deformable body having a head portion shaped to be positioned within a body cavity resulting from removal of a tumor, the body sized to receive a treatment agent;
a stem portion coupled to the body and sized to protrude out of the wound cavity and receive the treatment agent,
a conformance member that causes tissue in the vicinity of the head portion to become engaged with the head portion and optimize an amount of tissue treated by the treatment agent; and one or more markers coupled to the body.

5. The apparatus of claim 1, wherein the conformance member includes a device to provide a vacuum to adjacent tissue.

6. The apparatus of claim 1, wherein the treatment agent is selected from a physical or chemical agent.

7. The apparatus of claim 7, wherein the physical agent is selected from, radiation, thermal energy, electrical flow in a thermal element or laser light and a coolant.

8. The apparatus of claim 7, wherein the chemical agent is selected from, a biologically active compound, tissue coagulation agents and biologically active agents.

9. The apparatus of claim 7, further comprising:
an aspiration or vacuum device coupled to the head and configured to provide for removal of material.

10. The apparatus of claim 9, wherein the conformance member provides good contact with an exterior surface of the head.

11. The apparatus of claim 7, further comprising:
a coupling device coupled to the head and configured to provide retaining of the treatment agent in the body during a treatment session.

12. The apparatus of claim 1, wherein the head portion defines a non-deformable outer surface configured to be brought into firm engagement with tissue of the body cavity, the non-deformable body including one or more channels for receiving and locating a source of treatment agent in a predetermined position and provide that body cavity tissue positioned adjacent to the head receives a predetermined treatment dose.

13. The apparatus of claim 12, wherein the stem portion is coupled to the head portion and sized to protrude out of the body cavity and receive a treatment agent.

14. The apparatus of claim 12, wherein the body is made of a material that is substantially transparent to ionizing radiation.

15. The apparatus of claim 1, wherein the body is made of a material selected from an inert biocompatible material, metal, alloy and a bio-absorbable material.

16. The apparatus of claim 1, wherein the head potion includes openings or fluid-permeable materials to provide for suctioning or fluid exchange between the head and adjacent tissues

17. The apparatus of claim 1, wherein the body is made of a material that provides a non-distensible surface with small openings for improved conformance through suctioning or fluid exchange between the head and a wound surface.

18. The apparatus of claim 1, wherein the head portion is spherical or spheroidal.

19. The apparatus of claim 18, wherein the head portion has a diameter in the range of 1-150 millimeters.

20. The apparatus of claim 1, wherein the body include at least one channel sized to receive a guide tube for a physical agent.

21. The apparatus of claim 20, wherein the at least one channel terminates at a defined point in relation to a surface of the head portion.

22. The apparatus of claim 21, wherein the stem portion has a coupling at an end of the at least one channel that is remote from the head portion and adapted to receive a guide tube for positioning.

23. The apparatus of claim 22, wherein the coupling is configured to be coupled to a high dose rate, remotely controlled after-loading brachytherapy unit.

24. The apparatus of claim 22, wherein the coupling is configured to be coupled to an X Ray source.

25. The apparatus of claim 22, wherein the coupling is configured to be coupled to a miniaturized linear accelerator.

26. The apparatus of claim 22, wherein the coupling is configured to be coupled to a heat source.

27. The apparatus of claim 22, wherein the coupling is configured to be coupled to a cooling medium.

28. The apparatus of claim 22, wherein the coupling is configured to be coupled to a source of light.

29. The apparatus of claim 1, wherein the head portion includes at least a first channel to receive a physical or chemical agent, and at least a second channel that extends from the head portion to a coupling on the stem portion remote from the head portion.

30. The apparatus of claim 29, wherein the coupling at the at least second channel is configured to be coupled with a vacuum device.

31. The apparatus of claim 29, wherein the coupling at the at least second channel is configured to be coupled a reservoir for the administration of a substances.

32. The apparatus of claim 1, wherein at least one of the head portion or the stem portion is coated with a material selected from, an anticoagulant substance, a thrombolytic substance, a biocompatible adhesive substance, and ar biologically active substance.

33. The apparatus of claim 1, further comprising:
a mechanical device coupled to the head portion that improves tissue conformance.

34. The apparatus of claim 1, further comprising:
a marker that facilitates optical or imaging detection,

35. The apparatus of claim 1, further comprising:
a container that encloses a treatment site.

36. The apparatus of claims 35, wherein the container and the head portion physically interact

37. A method of administering a physical agent to human tissue comprising:
locating a source in an applicator body defining a rigid wound-engaging surface;
inserting the applicator body into a wound cavity to provide that a wound-engaging surface is in contact with tissue defining the wound cavity;
using a conformance member that causes tissue in the vicinity of the head portion to become engaged with the head portion;
leaving the applicator body in position in the wound cavity for a sufficient time to deliver a treatment dose of a physical agent to tissue adjacent the wound cavity and
removing the applicator body from the wound cavity.

38. The method of claim 37 wherein the applicator body is inserted through a wound opening that is closed about the applicator body to provide for tissue defining the wound cavity contacts a tissue-engaging surface of the applicator body.

39. The method of claim 37, wherein a source of the physical agent is introduced into the applicator body through a guide tube before or after insertion of the applicator body into the wound cavity.

40. The method of claim 37, wherein a source of the physical agent is an isotropic point source.

41. The method of claim 40 wherein the source is an isotope.

42. The method of claim 38 the treatment dose is a radiation dose delivered at a surface of the applicator in the range 5 to 30 Gy.

43. The method of claim 42 wherein the radiation dose delivered at the surface of the applicator is about 21 Gy.

44. The method of claim 37, wherein the physical agent is a source generating therapeutic X Rays.

45. The method of claim 37, wherein the physical agent is electromagnetic energy from an RF source.

46. The method of claim 37, wherein the physical agent is electromagnetic energy from a laser source.

47. The method of claim 37, wherein the physical agent is a coolant.

48. The method of claims 37, wherein the treatment dose is administered to a layer of tissue surrounding the applicator body with a thickness between 0 and 20 millimeters.

49. The method of claim 37, wherein the treatment dose is administered to a layer of tissue surrounding the applicator body with a thickness of about 10 millimeters.

50. The method of claim 37, wherein the treatment dose is delivered as a single dose.

51. The method of 37, wherein the treatment dose is delivered in multiple doses.

52. The method of claim 37, further comprising:
applying suction to draw the tissue into contact with the applicator body.

53. The method of claim 37, further comprising:
delivering at least one of a, medication or biologically active substance to improve contact between tissue and the applicator body.

54. The method of claim 37, further comprising:
delivering at least one of a, medication or biologically active substance to achieve a cytotoxic or cytostatic effect.

55. A system for treating walls of either naturally occurring cavities or cavities generated by the resection of tissue, comprising:
a body with a head portion shaped to be positioned within a body cavity resulting from removal of a tumor, the body sized to receive a treatment agent, the body including a first channel to receive a physical or chemical agent, and a second channel that extends away from a surface of the head portion;
a stem portion coupled to the body and sized to protrude out of the body cavity and receive the treatment agent, the first and second channels extend through the stem portion to a coupling on the stem portion remote from the head portion; and
a vacuum source configured to operate and connect with at least one of the first and second channels in the stem portion.
